(19) United States
(12) Patent Application Publication
(10) Pub. No.: US 2017/0007532 A1
(43) Pub. Date: Jan. 12, 2017

(54) Cosmetic Products for Reducing Hair Appearance

(71) Applicant: The Procter & Gamble Company, Cincinnati, OH (US)

(72) Inventors: Kojo Tanaka, Ashiya (JP); Maya Mitsumatsu, Singapore (SG); Michael Christopher Sabino, Cockeysville, MD (US)

(73) Assignee: The Procter & Gamble Company

(21) Appl. No.: 15/270,077

(22) Filed: Sep. 20, 2016

Related U.S. Application Data
(63) Continuation of application No. 13/905,236, filed on May 30, 2013.
(60) Provisional application No. 61/652,976, filed on May 30, 2012.

(51) Int. Cl. A61K 8/89; A61Q 7/02; A61Q 1/02; A61K 8/41; A61K 8/34

(52) U.S. Cl. CPC A61K 8/89; A61K 8/41; A61K 8/34; A61K 8/345; A61Q 1/02; A61Q 7/02; A61K 2800/43

(57) ABSTRACT

A cosmetic product for reducing the appearance of hair. The cosmetic product includes a composition made with at least one film forming polymer present in an amount sufficient to reduce the appearance of hair on a skin surface, and a dermatologically acceptable carrier. The product also includes indicia that communicates to a user that the composition is to be applied along the direction of hair growth on the area of skin where hair appearance reduction is desired.

### FIELD OF THE INVENTION

The present invention is directed, generally, to products and compositions for reducing the appearance of hair on a skin surface. More specifically, the present invention is directed to the use of a film-forming polymer to lay down hair against the surface of the skin so it is less noticeable.

### BACKGROUND OF THE INVENTION

Hair growth regulation often is desired to improve one's appearance and hygiene. Various methods and personal care products have been developed to remove unwanted hair, for example, shaving, electrolysis, waxing and depilatory creams. However, such conventional procedures frequently have drawbacks associated with them. Shaving, for instance, may cause nicks, cuts, rash and irritation and often leave undesirable stubble, as well as having to be carried out frequently. Electrolysis and laser hair removal can keep a treated area free of hair for prolonged periods of time, but can be either expensive, painful, and/or sometimes leave scarring. Waxing and plucking are painful and are poor options for shorter hair. Finally, depilatory creams, although effective, are messy to apply and typically are not recommended for frequent use due to their high irritancy potential. Therefore, these methods are not very welcomed by consumers, females especially, to managing unwanted facial hair though shaving is often used for facial hair removing. Shaving offers only short term hair removal, and must be repeated. For many consumers, continual growth of hair on the human scalp and face means that one must exert an on-going effort to hiding the hair that has grown out from the roots.

As an effort to mask perceived skin flaws such as spots and wrinkles, consumers tend to use make-up products such as foundations, which typically include pigments. Pigments are commonly used in make-up products to reduce or eliminate the appearance of perceived skin flaws and provide a natural looking finish. But, in some instances, pigments may accentuate the appearance of vellus hair on the face, which may be particularly undesirable for some people. Accordingly, it would be desirable to develop cosmetic compositions that enable consumers to manage and/or conceal unwanted hair.

### SUMMARY OF THE INVENTION

The present invention is related to a cosmetic product for reducing hair appearance in the skin comprising (a) a composition comprising at least one film forming polymer in an amount sufficient to provide hair appearance reducing effect on the skin, and dermatologically acceptable carrier; and (b) communication instructing to apply the composition along the direction of hair growth on the area of the skin where hair appearance reduction is desired.

### BRIEF DESCRIPTION OF THE DRAWING

FIG. 1 illustrates a grading sheet to rate the lay-down degree of hair.

### DETAILED DESCRIPTION OF THE INVENTION

The present invention describes products comprising compositions for providing reduced hair appearance on the skin and communication instructing how to apply the composition, and methods for reducing hair appearance in the skin.

All percentages are by weight of the total composition, unless specifically stated otherwise. All percentages and ratios used herein are by weight of the total composition, unless otherwise designated. All ranges are inclusive and combinable. The number of significant digits conveys neither a limitation on the indicated amount nor on the accuracy of the measurement. All numerical amounts are understood to be modified by the word “about” unless otherwise specifically indicated. All measurements are understood to be made at 25° C. and at ambient conditions, where “ambient conditions” means conditions under about one atmosphere of pressure and at about 50% relative humidity. All such weights as they pertain to listed ingredients are based on the active level and do not include carriers or by-products that may be included in commercially available materials, unless otherwise specified.

“Dermatologically acceptable carrier”, as used herein, means that the carrier is suitable for topical application to the skin, has good aesthetic properties, is compatible with the actives of the present invention and any other components, and will not cause any safety or toxicity concerns.

“Derivatives,” as used herein, includes but is not limited to, amide, ether, ester, amino, carboxyl, acetyl, and/or alcohol derivatives of a given compound.

“Hair,” as used herein, includes hair on any part of the body.

“Hair growth regulation compound,” as used herein, means a compound useful for regulating hair growth, and is understood not to encompass compounds such as depilatories.

“Hair lay down,” as used herein, means decrease of the angle of hair standing.

“Isomers,” as used herein, is understood to include D-isomers, L-isomers, and/or DL-isomers of a given compound.

“Keratinous tissue,” as used herein, means to keratin-containing layers disposed as the outermost protective covering of mammals (e.g., humans, dogs, cats, etc.) which includes, but is not limited to, skin, hair, etc.

“Regulating hair growth,” as used herein, means reducing, modulating, inhibiting, attenuating, retarding, and/or diminishing hair growth, and further may include regulating hair appearance, including maintaining a desired hair style for a longer period of time than would be expected without the use of a composition of the present invention. All relative terms used in connection with regulating hair growth and other benefits (such as softer, finer, reducing, less-noticeable, finer, etc.) are understood to mean that the benefit observed is relative to that which is observed or would be expected without the use of a composition described herein.

“Salts,” as used herein, includes but is not limited to sodium, potassium, calcium, ammonium, manganese, copper, and/or magnesium salts of a given compound.

The compositions suitable for the present invention may take a variety of final forms, non-limiting examples of which include lotions, creams, liquids, gels, mousses, emulsions, multi-phase emulsions, pastes, semi-solid forms, and tonics, and may be applied to the skin via a variety of means. The compositions herein may be formulated to be leave on products or rinse off products. For example, the compositions may be including in make-up products such as foundations, personal care products such as body washes and soaps and/or skin care products such as anti-aging creams and moisturizing lotions.

In some embodiments, the composition may include one or more polyols; one or more useful actives for enhancing the benefit(s) of the hair growth regulation (e.g., actives for reducing shaving frequency, improving the ease of hair removal, increasing shaving efficiency, making hair softer and finer, making hair less noticeable, slowing the re-growth of hair, reducing erythema and irritation to skin, maintaining a desired hair style for a longer period of time, maintaining a desired facial hair style for a longer period of time, reducing the appearance of pore size, and combinations thereof); and/or one or more chronic skin care actives.

### A. Composition

### Film Forming Polymer

The compositions used for the present invention comprise a film forming polymer. Without being limited by theory, it is believed that, by incorporating the film-forming polymers, the compositions can lay down hair during and after application of the compositions on the skin where the compositions are applied. Film forming polymers for the composition used for the present invention are those which can be used for skin, make-up and hair treatment compositions, and examples of suitable film forming polymeric materials include:
a) sulfopolyester resins, such as AQ sulfopolyester resins, such as AQ29D, AQ35S, AQ38D, AQ38S, AQ48S, and AQ55S (available from Eastman Chemicals);
b) polyvinylacetate/polyvinyl alcohol polymers, such as Vinex resins available from Air Products, including Vinex 2034, Vinex 2144, and Vinex 2019;
c) acrylic resins, including water dispersible acrylic resins available from National Starch under the trade name “Dermacryl”, including Dermacryl LT;
d) polyvinylpyrrolidones (PVP), including Luviskol K17, K30 and K90 (available from BASF), water soluble copolymers of PVP, including PVP/VA S-630 and W-735 and PVP/dimethylaminoethylmethacrylate Copolymers such as Copolymer 845 and Copolymer 937 available from ISP, as well as other PVP polymers disclosed by E.S. Barabas in the Encyclopedia of Polymer Science and Engineering, 2 Ed. Vol. 17 pp. 198-257;
e) high molecular weight silicones such as dimethicone and organic-substituted dimethicones, especially those with viscosities of greater than about 50,000 mPas;
f) high molecular weight hydrocarbon polymers with viscosities of greater than about 50,000 mPas;
g) organosiloxanes, including organosiloxane resins, fluid diorganopolysiloxane polymers and silicone ester waxes.

Examples of these polymers and cosmetic compositions containing them are found in PCT publication Nos. WO96/33689, WO97/17058 and WO98/18431 and U.S. Pat. No. 5,505,937 issued to Castrogiovanni et al., which are incorporated herein by reference.

Examples of high molecular weight hydrocarbon polymers with viscosities of greater than about 50,000 mPas include polybutene, polybutene terephthalate, polydecene, polycyclopentadiene, and similar linear and branched high molecular weight hydrocarbons. Preferred film forming polymers include organosiloxane resins comprising combinations of R₃SiO_{1/2} “M” units, R₂SiO “D” units, RSiO_{3/2} “T” units, SiO₂ “Q” units in ratios to each other that satisfy the relationship RₙSiO_{(4-n)/2} where n is a value between 1.0 and 1.50 and R is methyl. Note that a small amount, up to 5%, of silanol or alkoxy functionality may also be present in the resin structure as a result of processing. The organosiloxane resins must be solid at about 25° C. and have a molecular weight range of from about 1,000 to about 10,000 grams/mole. The resin is soluble in organic solvents such as toluene, xylene, isoparaffins, and cyclosiloxanes or the volatile carrier, indicating that the resin is not sufficiently crosslinked such that the resin is insoluble in the volatile carrier. Particularly preferred are resins comprising repeating monofunctional or R₃SiO_{1/2} “M” units and the quadrofunctional or SiO₂ “Q” units, otherwise known as “MQ” resins as disclosed in U.S. Pat. No. 5,330,747 to Krzysik, issued Jul. 19, 1994, incorporated herein by reference. In the present invention the ratio of the “M” to “Q” functional units is preferably about 0.7 and the value of n is 1.2. Organosiloxane resins such as these are commercially available such as trimethylsiloxysilicate/cyclomethicone D5 Blend available from GE Toshiba Silicone, Wacker 803 and 804 available from Wacker Silicones Corporation of Adrian Michigan, KP545 from Shin-Etsu Chemical and G. E. 1170-002 from the General Electric Company. In the present invention, by having film forming polymer mainly in the second layer, the film forming polymer will exist in a higher concentration at a localized area, and thereby forming a film of higher film intensity when applied to the skin, compared to the remainder of the composition. Such concentrated area of high film intensity provides improved adhesion of the entire composition to the skin. Namely, by providing the film forming polymer mainly in the second layer, the amount of film forming polymer included in the entire composition can be reduced, or if the same amount of film forming polymer is formulated in the second layer, an entire composition having improved adhesion is obtained.

A film forming polymer in the compositions of the present invention is present in an amount sufficient to provide hair appearance reducing effect on the skin, and the amount can be determined without undue experiment considering characteristics of a film forming polymer such as film forming capacity and impacts on skin sensory feel. Amount of a film forming polymer in the present invention may be from about 0.3% to about 10%, more preferably from about 1% to about 6%, and more preferably 1.5% to about 5% by weight of the composition.

### Dermatologically Acceptable Carrier

The compositions for the present invention also comprise a dermatologically acceptable carrier. The dermatologically acceptable carrier may be present in an amount of from about 50% to about 99.3%, preferably from about 60% to about 99%, more preferably from about 70% to about 98.5% by weight of the composition.

The carrier can be in a wide variety of forms. For example, emulsion carriers, including, but not limited to, oil-in-water, water-in-oil, water-in-oil-in-water, oil-in-water-in-oil emulsions, aqueous solution, water gel, oil dispersion and oil gel are useful herein. Oil in the carrier herein is understood to include silicone oil.

To maximize hair lay-down performance of the present composition, dermatologically acceptable carrier may be a carrier having an aqueous phase as a continuation phase such as oil-in-water and water-in-oil-in-water, aqueous solution, and water gel. However, dermatologically acceptable carrier may be selected different to consider other performance of the composition. The carrier may contain water in the range of from about 30% to about 80%, preferably from about 40% to about 60% by the weight of the composition.

### Optional Components

The compositions for the present invention may further contain additional components such as those conventionally used in topical products, e.g., for providing aesthetic or functional benefit to the composition or skin, such as sensory benefits relating to appearance, smell, or feel, therapeutic benefits, or prophylactic benefits (it is to be understood that the above-described required materials may themselves provide such benefits).

The CTFA Cosmetic Ingredient Handbook, Second Edition (1992) describes a wide variety of nonlimiting cosmetic and pharmaceutical ingredients commonly used in the industry, which are suitable for use in the topical compositions of the present invention. Such other materials may be dissolved or dispersed in the composition, depending on the relative solubilities of the components of the composition.

1. Polyol

The composition for the present invention may further comprise at least one polyol. Without being limited by theory, it is believed that polyols, in addition to its well known humectants function, are able to soften hair and thus hair becomes more susceptible for being laid down by a composition comprising a film forming polymer.

Polyols useful herein include polyhydric alcohols such as glycerin, 1,3-butylene glycol, propylene glycol, hexylene glycol, propane diol, ethylene glycol, diethylene glycol, dipropylene glycol, diglycerin, sorbitol, and other sugars which are in liquid form at ambient temperature. Also useful herein are water soluble alkoxylated nonionic polymers such as polyethylene glycol.

The amount of polyols in the compositions for the present invention can be determined depending on the desired characteristics of the product without undue experiment. In one embodiment, the polyol may be preferably comprised by weight of the composition at from about from about 5% to about 50%, preferably from about 7% to about 30% by weight of the composition, preferably from about 10% to about 25%, by weight of the composition.

### 2. Hair Growth Regulation Compounds

The compositions for the present invention may further comprise from about 0.001% to about 10%, preferably from about 0.01% to about 5%, and more preferably from about 0. 1% to about 1% of at least one hair growth regulation compound by weight of the composition. Preferably, the hair growth regulation compound is selected from the group consisting of butylated hydroxytoluene, butylated hydroxyanisole, hexamidine compounds, cetyl pyridinium chloride, green tea catechins, phytosterols, ursolic acid, difluoromethylornithine, alpha-methylornithine, 1,8-diaminooctane, bathocuprione, alanosine, arginine, extracts derived from soy plants, cinnamic acid, cinnamyl anthranilate, mannosamine and salts, derivatives and mixtures of any of the foregoing. As used herein, “hexamidine” includes hexamidine salts, any isomers and tautomers of such and is commercially available as hexamidine isethionate under the tradename Elastab® HP100 from Laboratoires Serobiologiques (Pulnoy, France).

The composition of the present invention may further comprise additional hair growth regulation compound selected from the group consisting of glyceryl dilaurate, including derivatives such as alpha, alpha-dilaurin and 1,2-dilauroyl-SN-glycerol apigenin, tetrahydrocurcumin, oleanolic acid, azelaic acid, sulforaphane, canavanine, pyridoxal-5-phosphate, phytic acid, tannic acid, grape seed extract, L-NAME, benzamidine, sodium butyrate, betulinic acid, polyornithine, polyarginine, fisetin, methyl-jasmonate, cis-jasmone caffeic acid phenethyl ester, delphinidin, ethyl abietate, esculetin, sorbic acid methyl ester, L-canaline, N-formyl-methionine, N-formyl-alanine, taurine, palmitoyl carnitine, undecanol, undecylenic acid, rutin, fusidic acid, phenyl pyruvic acid, L-isoleucine, phenyl glycine, silibinin, silymarin, L-ascorbic acid-6-palmitate, N-undecylenoyl-L-phenylalanine, and salts, derivatives and mixtures of any of the foregoing.

3. Pigments

i) Soft Focus Powder

The composition for the present invention may further comprise at least one soft focus powder. Soft focus powder is a pigment that is particularly effective in providing a soft focus effect to the composition, namely natural finish yet having good coverage for minimizing the appearance of skin troubles, when incorporated in a defined amount.

The soft focus powder useful herein includes polymethyl/methacrylate (PMMA), silica, hybrid pigments such as alumina treated mica, titanium dioxide treated talc, titanium dioxide treated mica, vinyl dimethicone/methicone silsesquioxane crosspolymer, alumina, barium sulfate and synthetic mica.

Preferred content level of soft focus powder is from about 0.1% to about 10%, more preferably from about 1% to about 5% by weight of the composition.

ii) Soft Focus Silicon Elastomer

The composition for the present invention may further comprise at least one soft focus silicone elastomer. A soft focus silicone elastomer is a crosslinked siloxane elastomer which is particularly effective in providing soft focus effect to the skin. In other words, when incorporated into a cosmetic product, the silicone elastomer can provide a natural finish and also have good coverage for minimizing the appearance of undesirable skin imperfections. Silicone elastomers suitable for use herein can be emulsifying or non-emulsifying crosslinked siloxane elastomers or mixtures thereof. The term “non-emulsifying” as used herein, defines crosslinked organopolysiloxane elastomers from which polyoxyalkylene units are absent. The term “emulsifying” as used herein, means crosslinked organopolysiloxane elastomer having at least one polyoxyalkylene (e.g., polyoxyethylene or polyoxypropylene) unit. Non-emulsifying elastomer useful in the present invention may be formed via crosslinking organohydrogenpolysiloxane with an alpha, omega-diene. Emulsifying elastomer herein includes polyoxyalkylene modified elastomer formed via crosslinking from organohydrogenpolysiloxane with polyoxyalkylene diene or organohydrogenpolysiloxane containing at least one polyether group crosslinked with an alpha, omega-diene. Emulsifying crosslinked organopolysiloxane elastomer can notably be chosen from the crosslinked polymer described in U.S. Pat. Nos. 5,412,004, 5,837,793, and 5,811,487. In addition, an emulsifying elastomer comprised of dimethicone copolyol crosspolymer (and dimethicone) is available from Shin Etsu under the tradename KSG-21.

Preferred content level of silicone elastomer is from about 0.1% to about 10%, more preferably from about 1% to about 5% by weight of the composition.

iii) Pigment Powder (Mention Make-Up Product)

The composition for the present invention may comprise from about 2% to about 45%, preferably from about 5% to about 30% of a pigment powder by weight of the composition, especially when the composition is a make-up composition. The pigments included in the pigment powder component herein are typically hydrophobic in nature, or hydrophobically treated. By keeping the level of pigment component low, the entire composition maintains flexibility to accommodate other components which provide spreadability, moisturization, and fresh and light feel. The species and levels of the pigments are selected to provide, for example, shade, coverage, good wear performance, and stability in the composition.

Pigments useful for the pigment component herein are inorganic and organic powder such as talc, mica, sericite, silica, magnesium silicate, synthetic fluorphlogopite, calcium silicate, aluminum silicate, bentonite and montmorillonite; pearl pigments such as alumina, barium sulfate, calcium secondary phosphate, calcium carbonate, coverage titanium oxide, finely divided titanium oxide, zirconium oxide, normal particle size zinc oxide, hydroxy apatite, iron oxide, iron titanate, ultramarine blue, Prussian blue, chromium oxide, chromium hydroxide, cobalt oxide, cobalt titanate, titanium oxide coated mica; organic powder such as polyester, polyethylene, polystyrene, methyl methacrylate resin, cellulose, 12-nylon, 6-nylon, styrene-acrylic acid copolymers, polypropylene, vinyl chloride polymer, tetrafluoroethylene polymer, boron nitride, fish scale guanine, laked tar color dyes, and laked natural color dyes. Such pigments may be treated with a hydrophobical treatment agent, including: silicone such as methicone, dimethicone, and perfluoroalkylsilane; fatty material such as stearic acid and disodium hydrogenated glutamate; metal soap such as aluminium dimyristate; aluminium hydrogenated tallow glutamate, hydrogenated lecithin, lauroyl lysine, aluminium salt of perfluoroalkyl phosphate, and aluminium hydroxide as to reduce the activity for titanium dioxide, and mixtures thereof.

4. Skin Active Agent

The compositions for the present invention may comprise a safe and effective amount of a skin active agent. The term “skin active agent” as used herein, means an active ingredient which provides a cosmetic and/or therapeutic effect to the area of application on the skin, hair, or nails. The skin active agents useful herein include skin lightening agents, anti-acne agents, emollients, non-steroidal anti-inflammatory agents, topical anaesthetics, artificial tanning agents, anti-microbial and anti-fungal actives, skin soothing agents, sun screening agents, skin barrier repair agents, anti-wrinkle agents, anti-skin atrophy actives, lipids, sebum inhibitors, sebum inhibitors, skin sensates, protease inhibitors, skin tightening agents, anti-itch agents, hair growth inhibitors, desquamation enzyme enhancers, anti-glycation agents, and mixtures thereof. When included, the present composition comprises from about 0.001% to about 20%, preferably from about 0.1% to about 10% by weight of the composition of at least one skin active agent.

5. Sunscreen Agent

The compositions for the present invention may comprise a safe and effective amount of a sunscreen agent such as a UV protection powder and a UV absorbing agent.

UV protection powder has a particle size of less than 100 nm, which size provide very little coverage effect to the skin. The composition of the present invention may comprise from about 0% to about 20%, preferably from about 0.1% to about 10% of a UV protection powder, such as micronized titanium dioxide and micronized zinc oxide. The powder included in the pigment component herein is typically hydrophobic in nature, or hydrophobically treated. Commercially available UV protection powder is titanium dioxide and methicone SI-TTO-S-3Z available from Miyoshi Kasei, titanium dioxide and dimethicone and aluminum hydroxide and stearic acid: SAST-UFTR-Z available from Miyoshi Kasei, Zinc oxide : Finex series available from Sakai Chemical Industry.

UV absorbing agent useful herein includes, for example, 2-ethylhexyl-p-methoxycinnamate (commercially available as PARSOL MCX), butylmethoxydibenzoyl-methane, 2-hydroxy-4-methoxybenzo-phenone, 2-phenylbenzimidazole-5-sulfonic acid, octyldimethyl-p-aminobenzoic acid, octocrylene, 2-ethylhexyl N,N-dimethyl-p-aminobenzoate, p-aminobenzoic acid, 2-phenylbenzimidazole-5-sulfonic acid, octocrylene, oxybenzone, homomenthyl salicylate, octyl salicylate, 4,4′-methoxy-t-butyldibenzoylmethane, 4-isopropyl dibenzoylmethane, 3-benzylidene camphor, 3-(4-methylbenzylidene) camphor, Eusolex™ 6300, Octocrylene, Avobenzone (commercially available as Parsol 1789), and mixtures thereof. A wide

When included, the present composition comprises from about 0.5% to about 20%, preferably from about 1% to about 15% by weight of the composition of a UV absorbing agent.

### B. Communication

The products of the present invention comprise communication to the consumer.

The manner in which the communication is conveyed to the consumer is non-limiting. By way of example only, the communication can be effected by known advertisement techniques, such as, television, internet and magazine advertisements. The communication may be a point-of-sale technique, such as, for example, a shelf and/or floor affixed communication. Additionally or alternatively, the communication may take the form of indicia (text, symbols, colors, shades, figures, and the like) disposed in and/or on the packaging of the personal care compositions. Additionally or alternatively, the communication may be provided in leaflets provided together with the compositions, or primary or secondary packages of the compositions. A primary package herein is understood as a package where the composition according to the present invention is directly placed in, and a secondary package herein is understood as a package which accommodates the primary package.

The communication may comprise images comparing the appearance of the skin of a consumer prior to and after use of the composition.

In one embodiment, the products of the present invention comprise communication instructing consumers to apply the composition along the direction of hair growth on the area of the skin where hair appearance reduction is desired. The direction of hair growth is the direction pointing from the root to the tip. The direction of hair growth may be easily determined by rubbing a hand or finger across the skin. The direction where less resistance is encountered is considered the direction of hair growth. The direction of hair growth may also be determined by visual observation.

In another embodiment, the products of the present invention comprise communication that the topical application of the composition for the present invention can reduce hair appearance.

In another embodiment, the products of the present invention comprise communication of dosage recommendation to provide hair appearance reduction on the skin where the composition for the present invention is applied.

### C. Methods of Use

### Application of the Composition

The present invention describes compositions useful for reducing hair appearance in the skin and methods of use thereof.

In one embodiment, a method according the present invention comprises at least one film forming polymer in an amount sufficient to provide hair appearance reducing effect on the skin, and dermatologically acceptable carrier.

Application of the compositions can occur through a variety of means, including with the fingers or hands, or by using an implement. Non-limiting examples of implements include a pad, cotton ball, applicator pen, roll-on applicator, and spray applicator

The composition may be applied to any part of the skin in need of reducing hair appearance and/or any additional benefit described herein.

### Application Regimens

The compositions of the present invention may be applied to the skin sequentially with a skin care product or make-up product. As an example, a cosmetic composition comprising at least one hair growth regulation compound is applied on the skin, and then the composition of the present invention is applied to the skin, wherein the cosmetic composition and the composition of the present invention differ from each other in terms of their compositions. As another example, the composition of the present invention is applied to the skin, and then, a cosmetic composition comprising at least one hair growth regulation compound applied to the skin, wherein the cosmetic composition and the composition of the present invention differ from each other in terms of their compositions. The regimen suggested herein can meet consumers demands in both long term hair minimization and short term hair appearance reduction by employing a cosmetic composition comprising at least one hair growth regulation compound, and a composition comprising a film forming polymer, and it can eventually provide enhanced hair appearance reducing effect.

In one embodiment, a method according to the present invention comprises a) applying to the skin a first composition comprising a film forming polymer in an amount sufficient to provide hair appearance reducing effect on the skin and dermatologically acceptable carrier; and b) applying to the skin a second cosmetic composition comprising at least one hair growth regulation compound and dermatologically acceptable carrier; and wherein the first and second compositions differ from each other in terms of their compositions. In the embodiment, the first composition is preferably applied along the direction of hair growth on the area of the skin.

In another embodiment, a method according to the present invention comprises a) applying to the skin a first cosmetic composition comprising at least one hair growth regulation compound and dermatologically acceptable carrier; and b) applying to the skin a second composition comprising a film forming polymer in an amount sufficient to provide hair appearance reducing effect on the skin and dermatologically acceptable carrier; and wherein the first and second compositions differ from each other in terms of their compositions. In the embodiment, the second composition is preferably applied along the direction of hair growth on the area of the skin.

In another embodiment, the present invention is for a kit comprising a) a first composition comprising a film forming polymer in an amount sufficient to provide hair appearance reducing effect on the skin, and dermatologically acceptable carrier; and b) a second composition comprising a hair growth regulation compound and dermatologically acceptable carrier, and wherein the first and second compositions are differ from each other in terms of their compositions.

### Test Method

### Hair Lay-Down Measurement

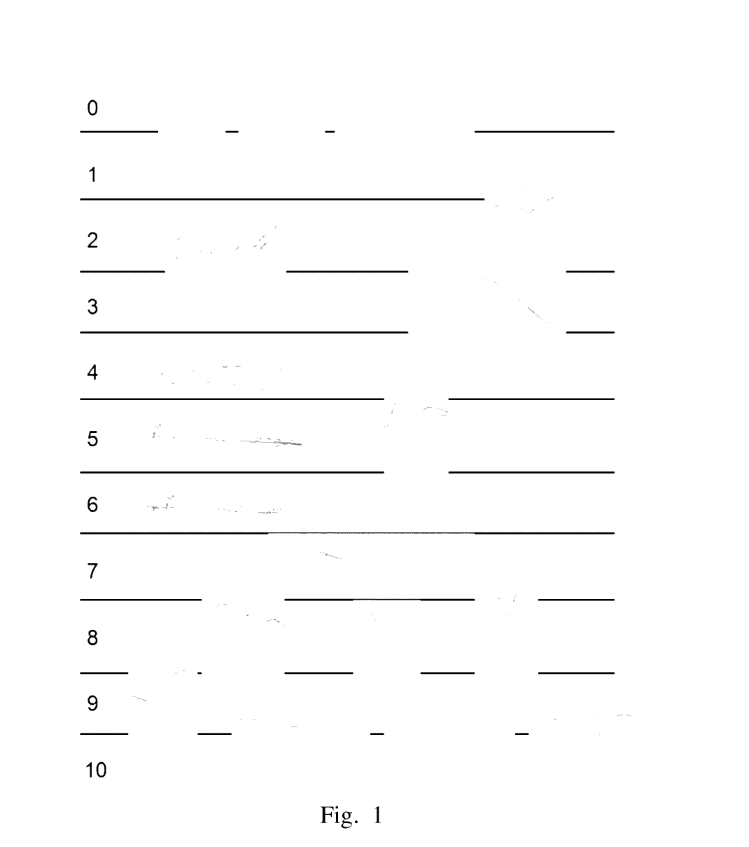

21 arm hairs with various lengths are implanted in artificial skin such as Bio Skin (model No. HO64-001) from Beaulax Co., Ltd. (Japan) Hair length is in the range of 0.5-1.8 cm after implanted in the artificial skin. Excess hairs at the backside of the artificial skin are cut and glue such as cyanoacrylate type instant glue is applied to the backside to adhere hairs on the artificial skin. 0.0125 g (0.0005 g/cm2) of a test sample is applied on the Bio Skin by a finger with finger sack until the sample is evenly distributed. 5 min later, each hair is rated based on a grading sheet of FIG. 1. An average hair lay down rate is calculated by dividing total of rating numbers by total numbers of hair. The number of hairs and the amount of a sample can be adjusted.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention.

Cosmetic compositions were prepared by conventional methods from the following components.

| | Ex. 1 | Ex. 2 | Ex. 3 | Ex. 4 | Ex. 5 | Com 1 |
|---|---|---|---|---|---|---|
| KF-7312J *¹ | 6.000 | 4.000 | | | | |
| Luviskol K17 *² | | | 2.000 | | 0.500 | |
| Daitosol 5000 SJ *³ | | | | 5.000 | | |
| Glycerin USP | 10.000 | 5.000 | 10.000 | | 10.000 | 10.000 |
| Propylene glycol | | | | 30.000 | | |
| Pentylene glycol | | 2.500 | | 3.000 | | 2.500 |
| 1,2 HEXANEDIOL | | 0.500 | | 2.000 | | 0.500 |
| DI Water | 52.500 | 48.659 | 54.467 | 37.341 | 55.967 | 43.659 |
| SA TTC-30 *⁴ | | 4.500 | 2.000 | | 2.000 | 4.500 |
| TTC-30 *⁵ | | | | 5.000 | | |
| Cyclomethicone D5 | 15.0000 | 11.1010 | | | | 15.1010 |
| Tridecyl isononanoate | 10.000 | 5.000 | 2.833 | | 2.833 | 5.000 |
| (WHICKENOL 153) | | | | | | |
| KF-6028 *⁶ | 2.000 | 1.500 | | | | 1.500 |
| Sorbitan isostearate | 0.500 | 1.500 | | | | 1.500 |
| (CRILL6) | | | | | | |
| Brij 72 *⁷ | | | 0.100 | | 0.100 | |
| Brij S721 *⁸ | | | 0.900 | | 0.900 | |
| Polysorbate 20 | | | | 1.000 | | |
| Octyl | | 2.000 | 7.000 | | 7.000 | 2.000 |
| methoxylcinnamate, | | | | | | |
| USP (UVINUL MC80) | | | | | | |
| Tocopheryl acetate | | | 0.200 | | 0.200 | |
| (DL) | | | | | | |
| Cetyl alcohol (APJ) | | | 0.200 | | 0.200 | |
| Stearyl alcohol | | | 0.600 | | 0.600 | |
| (LEROL C18) | | | | | | |
| Behenyl alcohol (High | | | 0.400 | | 0.400 | |
| stearyl) | | | | | | |
| BENTONE GEL VS- | | 1.500 | | | | 1.500 |
| 5PCV | | | | | | |
| RHEOPEARL KL2 *⁹ | 3.500 | 2.500 | | | | 2.500 |
| Ozokerite Wax | | | | | | |
| BHT | | 0.500 | | | | 0.500 |
| Ethyleparaben, NF | | | 0.200 | | 0.200 | |
| Propylparaben | | | 0.150 | | 0.150 | |
| SA/NAI-TR-10/D5 *¹⁰ | | 3.750 | | | | 3.750 |
| SA/NAI-Y-10/D5 *¹¹ | | 0.637 | | | | 0.637 |
| SA/NAI-R-10/D5 *¹² | | 0.327 | | | | 0.327 |
| SA/NAI-B-10/D5 *¹³ | | 0.196 | | | | 0.196 |
| SI-2 Yellow LL-100P *¹⁴ | | | 0.435 | | 0.435 | |
| SI-2 Red R-516P *¹⁵ | | | 0.240 | | 0.240 | |
| SI-2 Black BL-100P *¹⁶ | | | 0.096 | | 0.096 | |
| SA Titanium Dioxide | | | 5.544 | | 5.544 | |
| CR-50 *¹⁷ | | | | | | |
| SI TALC *¹⁸ | | | 0.835 | | 0.835 | |
| SI SILDEX H-52 *¹⁹ | | 2.000 | | | | 2.000 |
| SI TALC CT-20 *²⁰ | | 2.000 | | | | 2.000 |
| Silica Pearl P-4 *²¹ | | | 10.000 | 5.000 | 10.000 | |
| PGSS-22 TiO2 R250 *²² | | | | 8.330 | | |
| PGSS-22 Yellow | | | | 0.620 | | |
| No. 602P *²³ | | | | | | |
| PGSS-22 Red No. 211P | | | | 0.404 | | |
| Mix *²⁴ | | | | | | |
| PGSS-22 Black | | | | 0.205 | | |
| No. 710P *²⁵ | | | | | | |
| SEPIGEL 305 *²⁶ | | | 1.000 | 1.400 | 1.000 | |
| MAKIMOUSSE 12 *²⁷ | | | | 0.250 | | |
| Hexamidine | | 0.080 | | | | 0.080 |
| diisethionate | | | | | | |
| EDTA-2NA | 0.050 | | 0.050 | 0.050 | 0.050 | |
| Phenoxy Ethanol | 0.300 | 0.100 | | 0.400 | | 0.100 |
| Benzyl Alcohol | 0.150 | 0.150 | 0.500 | | 0.500 | 0.150 |
| Methylparaben | | | 0.250 | | 0.250 | |
| Total | 100.000 | 100.000 | 100.000 | 100.000 | 100.000 | 100.000 |

*¹ Trimethylsiloxysilicate (50%) and Cyclopentasiloxane (50%) form Shin-Etsu Chemical Co.
*² PVP (100%) from BASF Corporation
*³ Acrylates/Ethylhexyl Acrylate Copolymer (100%) from Daito Kasei Kogyo Co., Ltd.
*⁴ Titanium Dioxide and Aluminum Hydroxide and Talc and Magnesium Stearate and Dimethicone from Miyoshi Kasei, Inc.
*⁵ Titanium Dioxide and Aluminum Hydroxide and Talc and Magnesium Stearate from Miyoshi Kasei, Inc.
*⁶ PEG-9 Polydimethylsiloxyethyl Dimethicone from Shin-Etsu Chemical Co.
*⁷ Isosteareth-2 from Croda, Inc.
*⁸ Steareth-21 from Croda, Inc.
*⁹ Dextrin Palmitate from Chiba Flour Milling Company, Ltd.
*¹⁰ Titanium Dioxide and Cyclomethicone and Dimethicone and Disodium Stearoyl Glutamate and Aluminum Hydroxide from Miyoshi Kasei, Inc.
*¹¹ Iron Oxides and Cyclomethicone and Dimethicone and Disodium Stearoyl Glutamate and Aluminum Hydroxide from Miyoshi Kasei, Inc.
*¹² Iron Oxides and Cyclomethicone and Dimethicone and Disodium Stearoyl Glutamate and Aluminum Hydroxide from Miyoshi Kasei, Inc.
*¹³ Iron Oxides and Cyclomethicone and Dimethicone and Disodium Stearoyl Glutamate and Aluminum Hydroxide from Miyoshi Kasei, Inc.
*¹⁴ Iron Oxides and Methicone from Daito Kasei Kogyo Co., Ltd.
*¹⁵ Iron Oxides and Methicone from Daito Kasei Kogyo Co., Ltd.
*¹⁶ Iron Oxides and Methicone from Daito Kasei Kogyo Co., Ltd.
*¹⁷ Titanium Dioxide and Aluminum Hydroxide and Dimethicone from Miyoshi Kasei, Inc.
*¹⁸ Talc and from Methicone from Miyoshi Kasei, Inc.
*¹⁹ Silica and Methicone from Miyoshi Kasei, Inc.
*²⁰ Talc and from Methicone from Miyoshi Kasei, Inc.
*²¹ Silica from Presperse LLC
*²² Titanium Dioxide and Aluminum Hydroxide and Methoxy PEG-10 Propyltrimethoxysilane and Silica from Daito Kasei Kogyo Co., Ltd.
*²³ Iron Oxides and Methoxy PEG-10 Propyltrimethoxysilane and Silica from Daito Kasei Kogyo Co., Ltd.
*²⁴ Iron Oxides and Methoxy PEG-10 Propyltrimethoxysilane and Silica from Daito Kasei Kogyo Co., Ltd.
*²⁵ Iron Oxides and Methoxy PEG-10 Propyltrimethoxysilane and Silica from Daito Kasei Kogyo Co., Ltd.
*²⁶ Polyacrylamide and Water and C13-14 Isoparaffin and Laureth-7 from Seppic
*²⁷ Sodium Polyacrylate Starch from Daito Kasei Kogyo Co., Ltd.

As for Examples 1-3 and Comparison Examples land 2, in a suitable vessel, all hydrophilic and water soluble components except a thickener (SEPIGEL 305*26) were blended together, and mixed until all of the components were dissolved. In another vessel, all hydrophobic and oil soluble components except a thickener (RHEOPEARL KL2*9) were blended, and mixed until all of the components were homogenized. Mix above hydrophilic and hydrophobic ingredients for emulsification. A thickener was added to the obtained emulsion, and the emulsion was gently mixed. The emulsion was heated until 90C, then it was cooled down.

As for Example 4, in a suitable vessel, all hydrophilic and water soluble components except a thickener (SEPIGEL 305*26 and MAKIMOUSSE 12*27) were blended together, and mixed until all of the components were dissolved. Thickeners were added the mixture and the mixture was gently mixed.

### Measurement of Hair Lay-Down

Select examples were tested according to Hair Lay-Down Measurement, and provided the following average hair lay down rate. In the test, each sample

| | Ex. 1 | Ex. 2 | Ex. 3 | Ex. 4 | Ex. 5 | Com. 1 |
|---|---|---|---|---|---|---|
| Hair Lay-Down with | 1.3 | 1.1 | 1.8 | 2.4 | | |
| random application | | | | | | |
| Hair Lay-Down with | 1.9 | 1.5 | 3.0 | 5.1 | 1.9 | 0.9 |
| the direction of | | | | | | |
| hair growth application | | | | | | |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

Whereas particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A cosmetic product for reducing the appearance of hair on skin, the product comprising:
(a) a composition comprising a film forming polymer and a dermatologically acceptable carrier, the film forming polymer being present in an amount sufficient to provide a hair-appearance-reducing effect on the skin; and
(b) indicia that communicates to a user that the composition is applied along the direction of hair growth on the area of the skin where hair appearance reduction is desired.

2. The product according to claim 1, wherein said film forming polymer is selected from the group consisting of sulfopolyester resins, polyvinylacetate/polyvinyl alcohol polymers, acrylic resins, polyvinylpyrrolidones, PVP copolymers, PVP/dimethylaminoethylmethacrylate copolymers, dimethicones and organic-substituted dimethicones, hydrocarbon polymers, organosiloxane resins, and mixtures thereof, and wherein said film forming polymer is present at an amount of from about 0.3% to about 10% by weight of the composition.

3. The product according to claim 2, wherein said film forming polymer is an organosiloxane resin.

4. The product according to claim 1, wherein said composition further comprises a hair growth regulation compound, and wherein said hair growth regulation compound is selected from the group consisting of butylated hydroxytoluene, butylated hydroxyanisole, hexamidine compounds, cetyl pyridinium chloride, green tea catechins, phytosterols, ursolic acid, difluoromethylornithine, alpha-methylornithine, 1,8-diaminooctane, bathocuprione, alanosine, arginine, extracts derived from soy plants, cinnamic acid, cinnamyl anthranilate, mannosamine, and salts, derivatives and combinations of any of the foregoing.

5. The product according to claim 4, wherein said hair growth regulation compound is selected from the group consisting of butylated hydroxytoluene, butylated hydroxyanisole, hexamidine, cetyl pyridinium chloride, ursolic acid, and combinations thereof.

6. The product according to claim 1, wherein said composition further comprises a polyol, and wherein said polyol is present at an amount of from about 5% to about 50% by weight of the composition.

7. The product according to claim 1, wherein said composition comprises from about 30% to about 80% water by weight of the composition.

8. The product according to claim 1, wherein said composition is a leave-on make-up product that further comprises a pigment and at least one skin active agent.

9. The product according to claim 1, wherein said indicia is disposed on at least one of a primary package and a secondary package.

10. The product according to claim 1, wherein said film forming polymer is an organosiloxane resin; and said composition comprises
(a) a hair growth regulation compound selected from the group consisting of butylated hydroxytoluene, butylated hydroxyanisole, hexamidine, cetyl pyridinium chloride, ursolic acid, and combinations thereof,
(b) from about 5% to about 50% of glycerin by weight of the composition,
(c) from about 30% to about 80% water by weight of the composition,
(d) a pigment and
(e) a skin active agent;
wherein said indicia is disposed on at least one of a primary package and a secondary package of said composition.

11. A method for reducing hair appearance on skin comprising:
(a) identifying an area of skin where a reduction in hair appearance is desired; and
(b) applying to said area of skin a composition comprising a film forming polymer in an amount sufficient to provide hair appearance reducing effect on said area of skin, wherein said composition is applied along the direction of hair growth on the area of skin.

12. The method of claim 11, wherein the film forming polymer is organosiloxane resin and is at about 0.3% to about 10% by weight of the composition.

13. The method of claim 12, wherein the composition further comprises from about 5% to about 50% of glycerin by weight of the composition.

14. The method of claim 13, wherein the composition further comprises from about 30% to about 80% of water by weight of the composition.

15. The method of claim 14, wherein said composition is one selected from the group consisting of oil-in-water, water-in-oil, water-in-oil-in-water, oil-in-water-in-oil emulsions, aqueous solution, water gel and oil dispersion.

16. A method of reducing hair appearance on skin comprising:
a) identifying an area of skin where a reduction in hair appearance is desired;
b) applying a first composition to the area of skin, the composition comprising a film forming polymer in an amount sufficient to provide hair appearance reducing effect on the area of skin and a first dermatologically acceptable carrier; and
b) applying a second composition to the skin, the second composition comprising at least one hair growth regulation compound and a second dermatologically acceptable carrier, wherein the first and second compositions are different compositions.

17. The method of claim 16, wherein the film forming polymer of the first composition is an organosiloxane resin and is at about 0.3% to about 10% by weight of the first composition; and wherein the hair growth regulation compound of the second composition is selected from the group consisting of selected from the group consisting of butylated hydroxytoluene, butylated hydroxyanisole, hexamidine, cetyl pyridinium chloride, ursolic acid, and combinations thereof.
